(19) United States
(12) Patent Application Publication
(10) Pub. No.: US 2006/0067956 A1
(43) Pub. Date: Mar. 30, 2006

(54) Interface stabilisation of a product with 2 or more phases with a protein-polysaccharide complex

(75) Inventors: Eric Kolodziejczyk, Vevey (FR); Christophe Schmitt, Belmont S/Lausanne (CH)

Correspondence Address: BELL, BOYD & LLOYD LLC, P. O. BOX 1135, CHICAGO, IL, 60690-1135, US

(73) Assignee: Nestec S.A., Vevey (CH)

(21) Appl. No.: 10/529,245

(22) PCT Filed: Sep. 26, 2003
(86) PCT No.: PCT/EP03/10740

§ 371 Date: Mar. 24, 2005

(30) Foreign Application Priority Data
Sep. 27, 2002 (EP) 02021653.7

(51) Int. Cl. A61K 8/73; A61K 8/64

(52) U.S. Cl. 424/401; 424/70.13; 424/70.14

(57) ABSTRACT

The present invention concerns a product taken from the group consisting of a foam, an emulsion, a foamed emulsion, a dispersed emulsion and a foamed dispersion, wherein the interface water-air, water-oil or water-solid comprises a complex formed instantaneously by the mixture of at least a protein (or peptide) and at least a polysaccharide oppositely charged or the mixture of two proteins oppositely charged, said product being in a pH range within which the electrostatic interaction between both compounds oppositely charged occurs and wherein the total amount of protein and polysaccharide is comprised between 0.01 and 5% in weight.

### BACKGROUND OF THE INVENTION

The present invention concerns a two-phases or more product with enhanced stability.

A two-phases product, like a foamed product are very known on the market and appreciated by the consumer. An emulsion, like a mayonnaise, is also very spread on the market. For stabilizing the emulsion, an emulsifier is normally used, which is directly present in the bulk phase. The main drawback of this solution is the limitation of the diffusion of the emulsifier from the bulk to the interface resulting in a decrease in the final product stability. In the case of a foam, the control and design of texture is mainly achieved by adjusting the viscosity properties of the liquid bulk phase surrounding air bubbles (Walstra P. and De Roos A. L. (1993), Food Rev. Int., 9,503-525). To overcome the foam formation and foam stability problems, one generally combine surfactant molecules (phospholipids, fatty acids) together with tension active molecules (proteins). The former will initially decrease the interfacial area by adsorption at the interface, resulting in a high foam capacity. By contrast, the latter will form a viscoelastic layer around the bubbles, decreasing then the surface tension. This results in a higher stability of the foam. However, this combination has some drawbacks, since it requires the use of complex mixtures of surfactant and tension-active molecules. Moreover, it has been shown that both types of molecules are generally incompatible at the interface leading to interfacial phase separation and destabilisation (Mackie A. R. and al. (1999), J. Colloid Interf. Sc., 210, 157-166).

It is also possible to use protein-polysaccharide complexes to stabilize interfaces. This is the case for the U.S. Pat. No. 6,197,319, wherein a protein-polysaccharide complex is incorporated in a cosmetic composition, which is an emulsion. In this case, the complex is previously formed and then incorporated in the bulk. This is the same for the EP Patent No. 340'035: a microfragmented ionic polysaccharide/protein complex dispersion is formed to be used as fat substitute in food products, such as ice cream, salad dressings, dips, spreads and sauces. The U.S. Pat. No. 3,944,680 concerns a process for the preparation of an aqueous oil emulsion of prolonged storage life. In this case, the complex protein/polysaccharide is formed in the bulk, and there is a problem of diffusion of the complex from the bulk to the interface as already mentioned in the preceding paragraph.

Hence, these protein-polysaccharide complexes have been shown to form upon electrostatic attraction in well defined conditions of pH, ionic strength, protein to polysaccharide ratio, total biopolymer concentration, temperature or pressure (Schmitt C. et al. (1998), Crit. Rev. Food Sci. Nutr., 38, 689-753). In addition, various studies have demonstrated that these complexes exhibited better functional properties such as gelation, emulsification and foaming than that of the biopolymers alone. However, it is also known that the formation of complexes through electrostatic attraction between protein and polysaccharide lead to the associative phase separation phenomena (Piculell L. and Lindman B. (1992), Adv. Colloid Interf. Sci., 41, 149-178; Doublier J.-L. et al. (2000), Curr. Opinion Colloid Interf. Sci., 5, 202-214) or complex coacervation (Bungenberg de Jong H. G. (1936), La coacervation complexe et son importance en biologie, E. Fauré-Fremiet Ed, vol 1, Paris: Hermann et Cie). During associative phase separation which is a time dependent mechanism, the initial electrostatic protein-polysaccharide complexes interact ones with the others because of charge neutralisation to increase the electrostatic entropy of the system by release of counter-ions in the medium (Tolstoguzov V. B. (1997), Protein-polysaccharide interactions, S. Damodaran and A. Paraf Eds, Food Proteins and their Applications, pp 171-198, New York: Marcel Dekker Inc). Coming closer to the thermodynamic equilibrium, the complexes become insoluble and form liquid droplets called coacervates. These coacervates finally form a concentrated liquid phase at equilibrium with a very diluted phase containing mainly solvent (Mattisson K. W. et al. (1999), Macromol. Symp., 140, 53-76). The size of the successive entities formed ranged from tens of nanometers for the initial macromolecular complexes (Xia J. (1993) Macromolecules, 26, 6688-6690; Bowman W. (1997), Macromolecules, 30, 3262-3270) to hundreds of microns for the coacervates (Schmitt C. et al. (2001), Colloids and Surf. B: Biointerf., 20, 267-280). In terms of interfacial activity, it is well known that the coefficient of diffusion of the surface-active components is very important. Large molecular weight surface-active components (for example, protein-polysaccharide complexes) go very slowly at the interface to the contrary of low molecular weight surface-active components (sugar-esters, triglycerides). However, the former are much more effective for interfacial stabilisation (Dickinson E. and Galazka V. B. (1991), Food Hydrocolloids, 5, 281-296).

### SUMMARY OF THE INVENTION

It has been demonstrated that protein/anionic polysaccharide or basic protein/acidic protein mixtures are able to improve significantly the foaming properties as compared to the protein taken alone (Ahmed & Dickinson, 1991, Food Hydrocolloids, pages 395-402; Poole, 1989, International Journal of Food Science and Technology, pages 121-137 and GB 2134117 and GB 2179043). In the latter documents cited, no improvement of the foam stability is clearly described after mixing of the protein with another protein or polysaccharide. Thus, these papers refer only to increased foamability, which did not foresee any improvement of stability. An important point that is not mentioned in these documents is the effect of the time on the surface properties of the formed complexes. Since complexation is mainly due to electrostatic interactions between the two compounds, a charge neutralisation of the complexes arises with time (whatever the initial mixing ratio). It follows that more and more complexes are able to interact one with the other (no repulsion between the complexes), leading to an increase of the size of the complexes and to their progressive insolubilisation. These two phenomena are very detrimental for the stabilisation of an interface since they reduce the capability of the complexes to remain in solution and to migrate at the interface. This is the main reason why the industrial use of electrostatic complexes is scarce. Our invention allows circumventing these critical points in the use of protein/polysaccharide or protein/protein complexes. Since we are creating the complexes concomitantly with the interface (gas/liquid, gas/solid, or solid/liquid), they remain soluble and are small enough to go at the interface (with the help of the energy input within the system). Once at the interface, these complexes rearrange to form coacervates that effectively form a stabilising film and that can be detected at the interface within finished food products by means of microscopic histochemical techniques.

According to the present invention, it is possible to stabilise efficiently interfaces using protein-polysaccharide or protein-protein electrostatic complexes formed at the same time than the interface they have to stabilise, because they have smaller size and a higher diffusion coefficient. In this case, the protein-polysaccharide complexes will be soluble and with sufficiently low molecular weight to be at the interface. Based on these observations, the objective of the present invention is, in the case of a at least two phases product, to control the surface properties between said phases.

The present invention concerns a product taken from the group consisting of a foam, an emulsion, a foamed emulsion, a dispersed emulsion and a foamed dispersion, wherein the interface water-air, water-oil or water-solid comprises a complex formed instantaneously at said interface by the mixture of at least a protein (or peptide) and at least a polysaccharide oppositely charged or the mixture of two proteins oppositely charged, said product being in a pH range within which the electrostatic interaction between both compounds oppositely charged occurs and wherein the total amount of protein and polysaccharide is comprised between 0.01 and 5 % in weight.

The complex (or coacervate) in the product of the invention is formed instantaneously, directly during the preparation of said product and positioned directly at the interface water-air, water-oil and water-solid. The active compounds are in aqueous solutions or oil-in-water emulsions.

The purpose of the invention is to control the surface properties of the interfaces by using an ingredient mix of protein and polysaccharide or a mix of two proteins. The mix significantly enhances, in the case of foaming, the foam capacity, that means more foam is obtained and the foam stability (smaller air bubbles, less drainage). The mix can also be used for emulsions, and other at least two phases products.

The mix efficiency remains in the formation of electrostatic complexes under well defined conditions of pH (when electrostatic interaction occurs), temperature (from 0° C. to room temperature), with a ratio protein to polysaccharide or ratio of both proteins from 1:20 to 20:1 and total biopolymer concentration between 0.01 and 5% in weight. Under biopolymer, we understand the addition of the weight concentrations of protein and polysaccharide.

By using the complex in liquid form, in case of foam, it is possible to enhance the foam formation since the initially formed complex acts as surfactant. After the foam is formed, the complex further interacts one with the other to form the so-called coacervates. These coacervates exhibit high viscoelastic properties and by the way rearrange at the interface to form a viscoelastic film that stabilise the foam.

A product containing protein-polysaccharide or protein-protein complex obtained through ionic interaction is used to produce different types of interfaces from liquid dispersion. In a first step, the molecular complex acts as surfactant, so that the interfacial area is decreased. In a second step, the complex rearrange at the interface in order to form coacervates that spread around the air bubbles (or oil droplets), forming a viscoelastic film that stabilises the bubbles (or oils droplets) against destabilisation.

The instant formation of the protein-polysaccharide or protein-protein complex results in a characteristic structural signature combining both a typical structure and distribution of fats and a typical structure and composition of viscoelastic films at the water/air or water/oil or water/solid interfaces. This can be demonstrated with various microscopy techniques, in particular, by showing specifically the location of the protein(s) and the polysaccharide components within the interfaces: see below FIGS. 5 and 6.

In the case of a foamed product, the active compounds are in aqueous form, and the interface concerned is an air-water interface. In the case of an emulsion, the active compounds are in an aqueous phase, and the interface concerned is an oil-water interface. In the case of a foamed emulsion, both air-water and oil-water interfaces are concerned. In the case of a dispersed emulsion, both air-solid and oil-water interfaces are concerned. In the case of a foamed dispersion, both air-solid and water-air interfaces are concerned.

The protein of the product is taken from the group consisting of a protein from milk, soy, egg, meat, fish and plant. Under plant, we understand mainly cereals but also leguminosea. Most preferably, the protein is β-lactoglobulin, gelatin, α-lactalbumin, bovin serum albumin, soy globulin, soy protein, wheat protein, whey protein, soy protein, egg white protein.

The polysaccharide is taken from the group consisting of charged natural or synthetic polysaccharides. Most preferably, the polysaccharide is acacia gum, carboxy-methyl-cellulose, chitosan, xanthan, alginate, propylene-glycol alginate, carrageenans, low or high methoxylated pectins, arabinogalactans, rye arabinoxylans (Ax rye), wheat arabinoxylans (Ax wheat).

The product of the invention can be used either per se or in mixture with another product. In the case of a use in the food area, the final product is ice cream, a culinary product, chocolate, dessert, a dairy product, wafers, sponge cakes or a petfood product. It is also possible to use it in coffee creamer or dairy coffee creamer. In this case, the product of the invention is present in an amount of 10 to 100% of the final product in weight.

In the case of a use in the cosmetic or perfume area, the product is used in an amount comprised between 10 and 100% of the final product in weight.

The invention concerns further the processes for the preparation of the product of the invention. There are different possible products and the following description will consider all these possible ways.

In the case of a preparation of a foam product, the way of preparation is following:

a solution or a bulk mass of the at least one protein and a solution or a bulk mass of the at least one polysaccharide or a solution or a bulk mass of one protein and another solution or a bulk mass of one protein is injected with the air in a bulk mass or directly in the air in the case of two bulk masses using the multi-tube reactor described in FIG. 1. The bulk mass can be a dairy product, containing sugar or not, containing a living organism or not. In this case, the polysaccharide is preferably acacia gum and the protein is □L-lactoglobulin. The product has a final pH of around 4.2. The concentration of the protein and polysaccharide is around 0.01 to 5 % in weight. The ratio of protein to polysaccharide is around 20:1 to 1:20. The preparation is carried out at a temperature of around 4 to 50° C. In this case, the product obtained is the final product. The quantity of air injected is not critical and can vary between 1 0 and 700% of the product.

According to a second embodiment of the preparation of a foam product, the way of preparation is following: a solution of at least one protein and a solution of at least one polysaccharide or a solution of one protein and another solution of one protein are mixed together in the presence of air. This is the basic concept of the invention: the mixing of both active compounds creates per se the formation of the foam. If necessary, it is also possible to carry out a subsequent whipping. According to this way of proceeding, the product obtained is not the final product. It is then mixed with a preparation for ice cream, or with a preparation for a wafer.

In the case of a preparation of an emulsion the way of preparation is following:

A first part of an emulsion is stabilised with at least one protein, a second part of a second emulsion is stabilised with at least one polysaccharide or a second protein, and both emulsions are mixed together.

The lipid used for the first and the second emulsion is preferably palm oil, palm kernel, sunflower, safflower or olive oil or butterfat or butterfat or any of their mixtures. The main interest of this embodiment is the preparation of a mayonnaise, for example an egg-yolk free or low-fat mayonnaise. In this case, the protein is preferably β-lactoglobulin and the polysaccharide is acacia gum. The product has a final pH of around 4.2. The concentration of the protein and polysaccharide is around 0.01 to 5% in weight. The preparation is carried out at a temperature at which the fat is liquid. In this case, the product obtained is the final product. This is a non foamed product.

It is also possible, according to a second embodiment of the preparation of an emulsion to have a foamed emulsion. In this case, a solution of at least one protein and a solution of at least one polysaccharide or a solution of one protein and another solution of one protein are mixed together in the presence of air. This foamed product is then incorporated in an emulsion to obtain for example a foamed mayonnaise. In this case, the protein is β-lactoglobulin, the polysaccharide is acacia gum and the lipid phase is sunflower oil or olive oil. The overrun of whipping is around 10 to 700%.

According to a further embodiment of the invention, it is possible to prepare directly a foamed emulsion. In this case, the way to proceed is following:

a bulk product is prepared using a first part of an emulsion which is stabilised with at least one protein, a second part of an emulsion is stabilised with at least one polysaccharide or a second protein, both emulsions are mixed together and diluted in the bulk product, then a new dispersion of the protein with a new dispersion of the polysaccharide are injected with air (using the foaming device described on FIG. 1) in the bulk product to form the foamed bulk product.

In this case, the bulk product is preferably a mix for ice cream. The lipid used for the emulsion is preferably sunflower oil, palm oil, palm kernel oil or milk fat. The protein used is preferably β-lactoglobulin and the polysaccharide is acacia gum. The amount of both protein and polysaccharide is around 0.01 to 5% in weight. The pH is around 4.2. The final product is then either frozen by a static freezing or by a dynamic freezing.

According to a further embodiment, a dispersed emulsion can be prepared. The way of preparation is following:

a first part of an emulsion is stabilised with at least one protein, a second part of an emulsion is stabilised with the at least one polysaccharide or a second protein, and both emulsions are mixed together, the obtained final emulsion being then mixed with a base comprising particles. In a preferred embodiment, the base comprising particles is a chocolate base containing for example sugar, cocoa particles, milk powder, lecithine and other aroma.

The way of producing is following:

A first part of an emulsion is stabilised with at least one protein, a second part of a second emulsion is stabilised with at least one polysaccharide or a second protein, and both emulsions are mixed together.

The lipid used for the first and the second emulsion is preferably palm oil, palm kernel, sunflower, safflower or olive oil or butterfat or butterfat or any of their mixtures. The main interest of this embodiment is the preparation of a mayonnaise, for example an egg-yolk free or low-fat mayonnaise. In this case, the protein is preferably β-lactoglobulin (BLG) and the polysaccharide is acacia gum (AG). The product has a final pH of around 4.2. The concentration of the protein and polysaccharide is around 0.01 to 5% in weight. The preparation is carried out at a temperature at which the fat is liquid. This emulsion product is then mixed with a bulk composed by lecithin, sugar and cocoa particles to obtain a chocolate base.

According to a last embodiment of the invention, a foamed dispersion is prepared. The way of producing is following:

a solution of the at least one protein and a solution of the at least one polysaccharide or a solution of one protein and another solution of one protein is injected with the air in a bulk of dispersed particles.

A typical example of a foamed dispersion is the preparation of a sorbet. In this case, a solution of the at least one protein and a solution of the at least one polysaccharide or a solution of one protein and another solution of one protein is injected with the air in a bulk mass. In this case, the bulk mass does not contain fat, but more a fruit, or juice or fruit puree.

The bulk dispersed particles can be a yoghurt preparation, or another acidic gel with bacteria or with a gel obtained by chemical acidification or heat treatment of gelling polysaccharides or proteins.

It is also possible to proceed according to a second way: a solution of at least one protein and a solution of at least one polysaccharide or a solution of one protein and another solution of one protein are mixed together in the presence of air. The bulk of dispersed particles is then mixed with this foam to obtain the foamed dispersion.

For this last embodiment, it is possible to consider the preparation of wafers or caramel.

The invention concerns further the device for carrying out the process of the invention. This device comprises on a frame:

a first pipe for the arrival of an emulsion or dispersion with a protein

a second pipe for the arrival of the gas

a third pipe for the arrival of an emulsion or dispersion with a polysaccharide,

these three pipes arriving on a main channel, being disposed perpendicular and staggered along said main channel, the first pipe forming the central pipe on the main channel, the second pipe forming the intermediate pipe on the main channel and the third pipe forming the external pipe on the main channel and wherein the outlet on the main channel of the central and the intermediate pipe are staggered.

A further object of the present invention is to have a product, wherein the stability of its structure during storage or heat shock is kept practically unchanged. Under storage in the present description, we understand a storage of one to twelve months. Considering the case of ice cream, by practically unchanged, we understand that the volume of air bubbles does not decrease much by taking out and putting again said ice cream several time in the freezer. The consumer has therefore a product, quality remains practically unchanged during its whole storage. This is the same for the other products considered in the present patent.

A further advantage of the present invention is that in the case of a co-emulsion, no emulsifier is needed in the product according to the invention.

Additional features and advantages of the present invention are described in, and will be apparent from, the following Detailed Description of the Preferred Embodiments and the figures.

### BRIEF DESCRIPTION OF THE FIGURES

FIG. 1 shows a perspective view of the device (triple needle) according to the invention and

FIG. 2 is an enlarged view of the outlet of the device.

FIG. 3 is a microscopical view of the air bubbles in the ice-cream according to example 2 further to their dispersion in the medium used for the quantitative analysis,

FIG. 4 is a graph of the cumulative volume distribution of air bubbles analysed in example 2,

FIG. 5 show confocal scanning laser microscopy of products of example 2,

FIG. 6 show confocal scanning laser microscopy of products of example 2, at a higher resolution and

FIG. 7 shows the interface of an air bubble where coacervates were formed instantaneously at the interface in a model system of an air bubble in a biopolymer mixture solution.

### DETAILED DESCRIPTION OF THE PREFERRED EMBODIMENTS

The injection system is on a frame (4). In this frame, there are 3 pipes. The pipe (1) is used for the arrival of the first dispersion with the protein, the pipe (2) is used for the entrance of the gas, that is the air and the pipe (3) is used for the arrival of the second dispersion with the polysaccharide. The 3 pipes of the device arrive on a main channel (8): the are disposed perpendicularly and staggered along said main channel (8). This device is used for the different examples in the specification and hereunder.

The important feature is that at the point of the mixing of the gas with the first and the second dispersion the outlet of pipes (5) and (6) are staggered as shown on FIG. 2. This allows a reaction time between the components of the mixture.

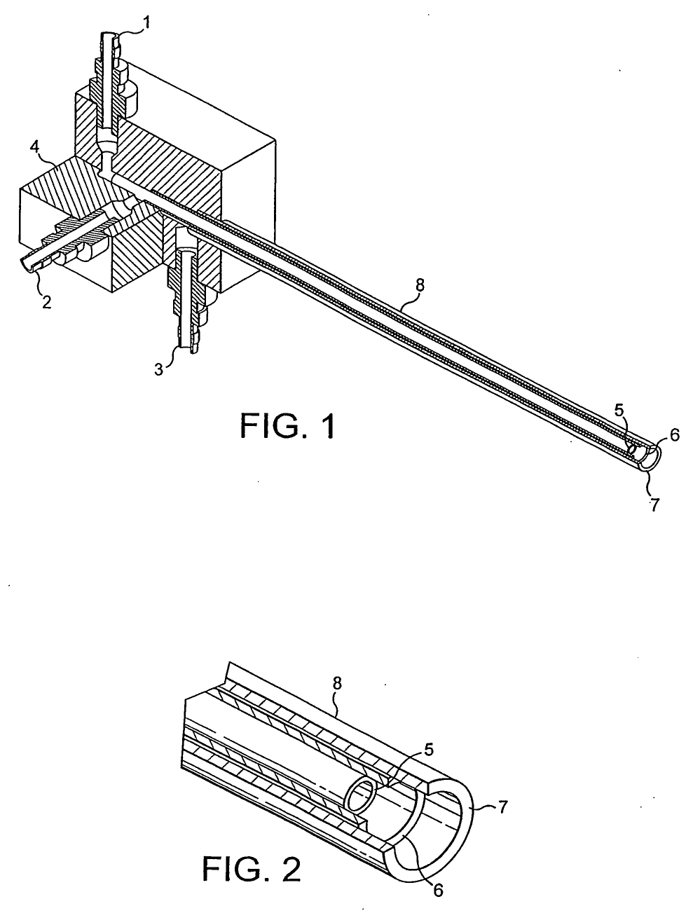

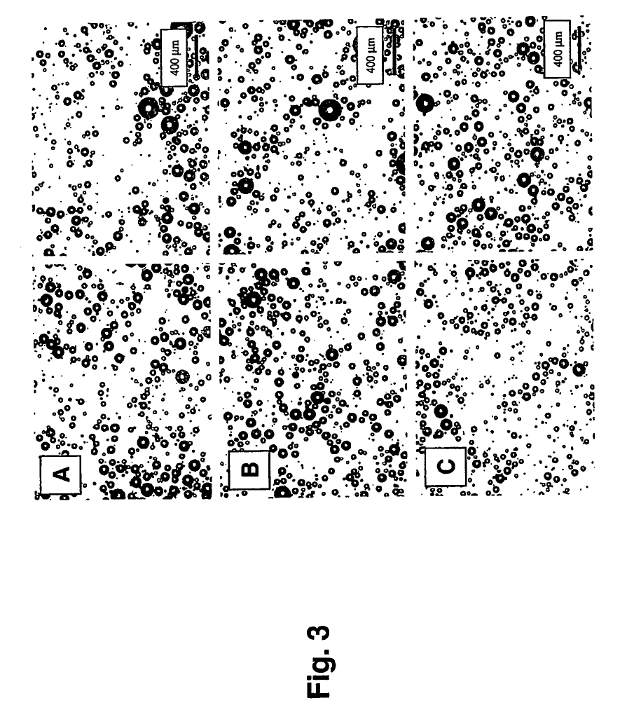

FIG. 3 is the air bubbles dispersion found after whipping to 100% overrun at +4° C. the ice-cream recipe described in example 2 using a Mondomix apparatus. Picture A is a classical mixture (10% non-fat milk solids) that did not contained the β-lactoglobulin and the acacia gum. The shearing speed was set to 800 rpm. Picture B is the same recipe but part of the non-fat milk solids was replaced by 2.5% whey protein isolate and 1.25% acacia gum. Here also shearing speed was 800 rpm. Picture C was taken after aeration of the recipes described in example 2 using the injection device (FIG. 1) coupled with the whipping device (Mondomix). The air bubble size is the same than in A and B, but the shearing rate is only 400 rpm. These results clearly demonstrated the highest foamability of the ice cream recipe containing protein-polysaccharide complexes instantaneously produced at the interface.

The air bubble analysis was carried out according to the procedure briefly described below:

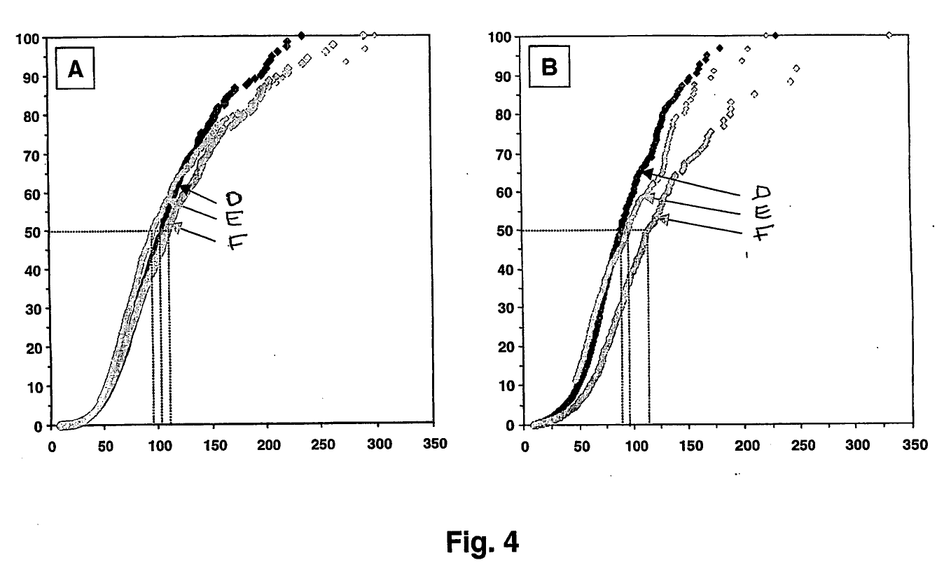

An aliquot of the product is weighed and dispersed in a medium of high viscosity. The composition of the dispersing medium is designed to stabilize the air bubbles. The dispersion is submitted to an automated quantitative image analysis. The same procedure is taken for FIG. 4.

FIG. 4 represents the cumulated volume size distributions of the recipes corresponding to the previous control (D), BLG/AG control (E) and triple needle BLG/AG (F) recipes described before (A) and after (B) heat shock treatment. The graphs represent the cumulative distribution of air bubbles in percent of the total volume of air analysed (x axis gives the air bubble diameter in mm and y axis gives in % the cumulated air volume analysed). Samples were equilibrated for one week at −40° C. before bubbles were measured. Heat-shock was achieved with 7 days cycle of temperature variation between −20 and −8° C. Each increase or decrease of temperature lasted 12 hours. Bubble size distribution was achieved by image analysis of the bubbles taken from 4 repetitions of the same sample after dispersion in a viscous glycerol containing medium. It is clear that the initial size distributions are very similar, which corroborates the previous microscopical observations on the bubbles (the sample produced with our invention required 2 times less energy input). After heat shock, both the control and BLG/AG control showed a shift of the size distribution to smaller air bubbles, which is in fact due to a loss of the largest air bubbles. Difference in air bubble content is the area between the initial and the heat-shock curves. Very interestingly, the curve corresponding to our invention did not move at all. This reveals the very high heat-shock stability of the sample.

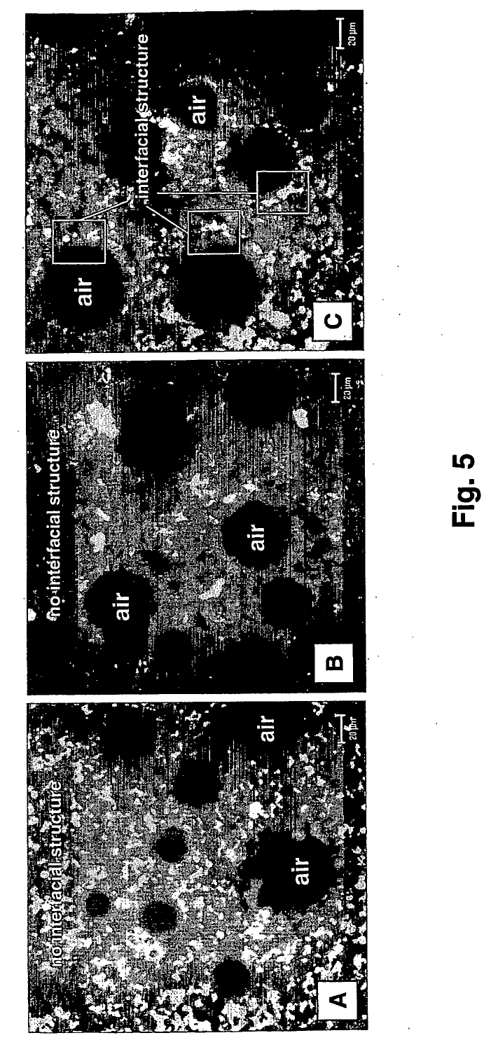

FIG. 5 shows the confocal scanning laser microscopy pictures of the three ice-cream products after 7 days equilibration at −40° C. The protein was coloured by the rhodamine 6G reagent that is known to specifically label protein through hydrophobic interactions. The confocal microscopy is carried out as follows: an aliquot of the product is put into a 2 mm deep covet and covered with 100 μl of a 10⁶ M aqueous solution of Rhodamine 6G. After melting, the melting is covered with a glass cover slide and examined by confocal microscopy. On the figure, the views are following: (A) control recipe; (B) BLG/AG control recipe; (C) recipe according to our invention. From the observation of the pictures, the structuration of the air bubble interface with proteins using our invention is clear as compared to the two controls. In these two latter products, proteins are somewhat distributed randomly within the matrix.

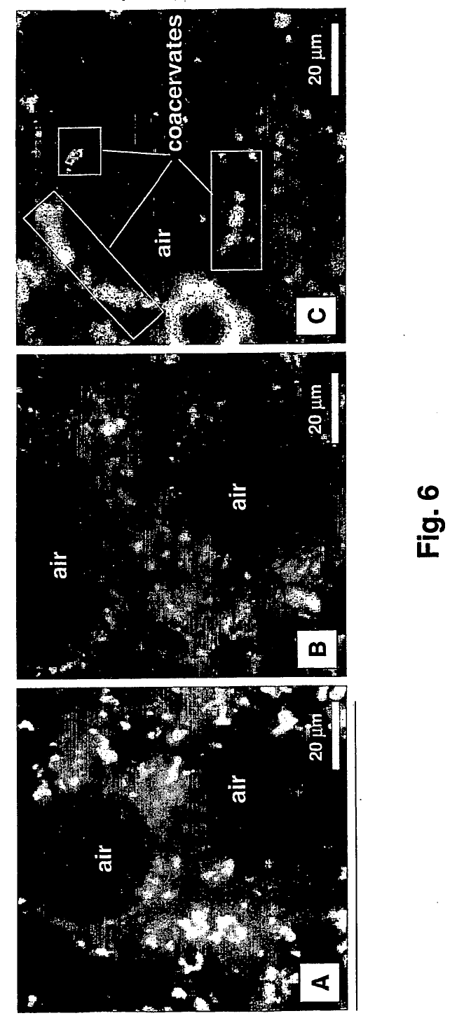

FIG. 6 represents the thin section micrographs of the three ice-cream products after Rhodamine 6G coloration according to the same procedure as for the preceding figure. On this figure, the views are following: (A) control recipe; (B) BLG/AG control recipe; (C) recipe according to hour invention. The presence of specific structures around the air bubbles has to be noted. To our opinion, these are coacervates made from the interfacial interaction between the primary formed BLG/AG complexes.

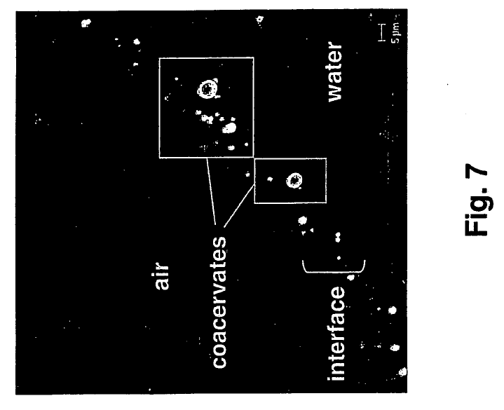

FIG. 7 shows the confocal scanning laser micrograph of the interface of an air bubble made in a just made mixture of β-lactoglobulin 2.5% and acacia gum 1.25% at pH 4.2 in water. β-lactoglobulin was covalently labeled with fluoresceine isothiocyanate. The coacervates present at the interface are comparable to those observed in the final ice cream product obtained according to our invention (FIG. 6C).

### EXAMPLES

The following examples illustrate some applications of the present invention.

### Example 1

An concentrated emulsion is obtained preparing first an emulsion by mixing during 5 minutes using a mixer 0.74% whey protein isolate, 66% sunflower oil and water at pH 4.2 by addition of lactic acid. A second emulsion is then prepared by mixing during 5 minutes using a mixer 0.23% acacia gum powder, 66% sunflower oil and water at pH 4.2 by addition of lactic acid. The final concentrated emulsion is obtained by mixing the two previously prepared emulsions at a 1:1 weight ratio and mixing during 10 minutes using a mixer or a high shear pump.

### Example 2

An ice cream mix is prepared from 9% palm-palm kernel oil, 5% non-fat milk solids, 5% whey protein isolate, 17% sucrose, 0.4% of a stabiliser blend containing hydrocolloids such as locust bean gum, guar, carrageenans, carboxymethylcellulose, water and emulsifiers. The pH of this first mix is adjusted to pH 4.2 by addition of citric acid. A second mix is prepared from 9% palm kernel oil, 7.5% non-fat milk solids, 2.5% acacia gum powder, 14% sucrose, 0.4% of a stabiliser blend containing hydrocolloids such as locust bean gum, guar, carrageenans, carboxymethylcellulose, water and emulsifiers. The pH of this second mix is adjusted to pH 4.2 by addition of citric acid. Both mix are then homogenized at 100 bars using a homogenizer and then pasteurised. After maturation at 4° C., the two ice cream preparations were mix together at a 1:1 mixing ratio and aerated at −6° C. to +4° C. using the processing device described on FIG. 1 coupled with a shearing device (Hoyer freezer or Mondomix whipper) to obtain an overrun of 100%. The mix was then poured in molds and hardened at a temperature of −40° C.

### Example 3

A sour cream mousse is prepared from 50% milk cream (30% fat), 10% non-fat milk solids, 5% whey protein isolate, 8% sucrose, water and emulsifiers. The pH of this first recipe is adjusted to pH 4.3 by addition of citric acid. A second recipe is prepared from 50% milk cream (30% fat), 10% non-fat milk solids, 2.5% acacia gum powder, 8% sucrose, 0.4% of a stabiliser blend, water and emulsifiers. The pH of this second recipe is adjusted to pH 4.3 by addition of citric acid. Both preparations are then homogenized at 80 bars using a homogenizer and then pasteurised. The two preparations were mix together at a 1:1 mixing ratio and aerated at +4° C. using the processing device described on FIG. 1 coupled with a Mondomix whipper to obtain an overrun of 100%. The aerated sour cream mousse was then poured in molds and stored at +4° C.

### Example 4

A wafer recipe is obtained by mixing using a mixer 50% of a wheat flour containing 70% of starch and 4% of a whey protein concentrate with water and adjusting the pH to 4.2 by addition of lactic acid. Another recipe is obtained by mixing using a Hobbard mixer 50% of a wheat flour containing 70% of starch and 1.6% of an acacia gum powder with water and adjusting the pH to 4.2 by addition of lactic acid. Both recipes are then mixed together at a 1:1 mixing ratio and aerated using the mixing device described on FIG. 1 coupled with Hobbard mixer equipped with a thermostated bowl. This aerated wafer base is then poured in-between baking plates at 150° C. and cooked to obtain wafers.

### Example 5

A liquid dairy coffee creamer recipe is obtained by mixing and high-pressure homogenization (400 bars+80 bars) 10% maltodextrin DE21, 15% hydrogenated palm oil, 3% whey protein isolate with water at 50° C. and adjusting the pH to 4.2 by addition of hydrochloric acid. Another recipe is obtained by mixing and high-pressure homogenization (400 bars+80 bars) 10% maltodextrin DE21, 15% hydrogenated palm oil, 3% acacia gum powder with water at 50° C. and adjusting the pH to 4.2 by addition of hydrochloric acid. Both recipes are then mixed together at a 1:1 mixing ratio using a high-shear mixer equipped with a thermostated bowl at 50° C.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A product selected from the group consisting of a foam, an emulsion, a foamed emulsion, a dispersed emulsion and a foamed dispersion, wherein the interface water-air, water-oil or water-solid comprises a complex formed instantaneously at the interface by the mixture of at least one of a protein or peptide and at least one of a polysaccharide oppositely charged or the mixture of two proteins oppositely charged, the product having a pH range within which the electrostatic interaction between both compounds oppositely charged occurs and wherein the total amount of protein and polysaccharide is between 0.01 and 5% by weight.

2. A product according to claim 1, wherein the protein is selected from the group of proteins consisting of milk, soy, egg, meat, fish and plant.

3. A product according to claim 1, wherein the protein is selected from the group consisting of β-lactoglobulin, whey protein, soy protein, egg white protein and wheat protein.

4. A product according to claim 1, wherein the polysaccharide is selected from the group consisting of gums, hydrocolloids and stabilizers.

5. A product according to claim 1, wherein the polysaccharide is selected from the group consisting of acacia gum, arabinogalactans, Ax rye, Ax wheat, carboxy-methyl-cellulose, chitosan, xanthan gum, and β-glucan.

6. A product according to any of claim 1, wherein the ratio protein to polysaccharide or protein to protein is between 1:20 and 20:1.

7. A method of producing a product selected from the group consisting of ice-cream, low-fat mayonnaise, culinary, chocolate, dessert, wafers, sponge cakes, dairy and petfood area comprising the step of using a product comprising a product selected from the group consisting of a foam, an emulsion, a foamed emulsion, a dispersed emulsion and a foamed dispersion, wherein the interface water-air, water-oil or water-solid comprises a complex formed instantaneously at the interface by the mixture of at least one of a protein or peptide and at least one of a polysaccharide oppositely charged or the mixture of two proteins oppositely charged, the product having a pH range within which the electrostatic interaction between both compounds oppositely charged occurs and wherein the total amount of protein and polysaccharide is between 0.01 and 5% by weight in an amount of between 10 and 100% of the final product by weight.

8. A method of producing a product selected from the group consisting of cosmetics and perfumes, comprising the step of using a product comprising a product selected from the group consisting of a foam, an emulsion, a foamed emulsion, a dispersed emulsion and a foamed dispersion, wherein the interface water-air, water-oil or water-solid comprises a complex formed instantaneously at the interface by the mixture of at least one of a protein or peptide and at least one of a polysaccharide oppositely charged or the mixture of two proteins oppositely charged, the product having a pH range within which the electrostatic interaction between both compounds oppositely charged occurs and wherein the total amount of protein and polysaccharide is between 0.01 and 5% by weight in an amount of between 10 and 100% of the final product by weight.

9. A process for the preparation of a foam product, comprising the step of using a solution or a bulk mass of the at least one protein and a solution or a bulk mass of at least one polysaccharide or a solution or a bulk mass of one protein and another solution or a bulk mass of one protein and is injected with the air in a bulk mass or directly in the air.

10. A process for the preparation of a foam product, comprising the steps of mixing a solution of at least one protein and a solution of at least one polysaccharide or a solution of one protein and another solution of one protein together in the presence of air.

11. A process for the preparation of an emulsion, comprising the steps of stabilising a first part of an emulsion with the at least one protein, stabilising a second part of an emulsion with the at least one polysaccharide or a second protein, and mixing the emulsions together.

12. A process for the preparation of a foamed emulsion, comprising the steps of preparing a bulk product, stabilizing a first part of an emulsion with at least one protein, stabilizing a second part of an emulsion with at least one polysaccharide or a second protein, mixing both emulsions together and diluting in the bulk product, then creating a foam product by injecting a new solution of the protein with a new solution of the polysaccharide with air in the bulk product to form the foamed bulk product.

13. A process for the preparation of a dispersed emulsion, comprising the steps of stabilizing a first part of an emulsion with at least one protein, stabilizing a second part of an emulsion with the at least one polysaccharide or a second protein, and mixing both emulsions together, mixing the obtained final emulsion with a base comprising particles.

14. A process for the preparation of a foamed dispersion, comprising the steps of producing a product wherein a solution of the at least one protein and a solution of the at least one polysaccharide or a solution of one protein and another solution of one protein is injected with the air in a bulk of dispersed particles.

15. A device for preparing an emulsion or dispersion comprising on a frame:
a first pipe for receiving an emulsion or dispersion with a protein;
a second pipe for receiving a gas;
a third pipe for receiving an emulsion or dispersion with a polysaccharide,
wherein the three pipes are coupled to a main channel, being disposed perpendicular and staggered along said main channel, the first pipe forming the central pipe on the main channel, the second pipe forming the intermediate pipe on the main channel and the third pipe forming the external pipe on the main channel and wherein the outlet on the main channel of the central and the intermediate pipe are staggered.

16. The device of claim 12, wherein the emulsion is a foamed emulsion.

17. The device of claim 12, wherein the emulsion is a dispersed emulsion.

18. The device of claim 12, wherein the dispersion is a foamed dispersion.

19. The device of claim 12, wherein the gas is air.

20. The device of claim 12, wherein the device can inject air into a solution or bulk product.
